(19) United States
(12) Patent Application Publication
(10) Pub. No.: US 2018/0243208 A1
(43) Pub. Date: Aug. 30, 2018

(54) AUGMENTATION OF PERSONALIZED TUMOR SPECIFIC ADAPTIVE IMMUNITY THROUGH EXTRACORPOREAL REMOVAL OF IMMUNE BLOCKING FACTORS

(71) Applicant: Immunicom, Inc., San Diego, CA (US)

(72) Inventors: Thomas Ichim, San Diego, CA (US); Amir Jafri, San Diego, CA (US); Steven F. Josephs, San Diego, CA (US)

(73) Assignee: Immunicom, Inc., San Diego, CA (US)

(21) Appl. No.: 15/908,620

(22) Filed: Feb. 28, 2018

Related U.S. Application Data
(60) Provisional application No. 62/465,114, filed on Feb. 28, 2017.

(51) Int. Cl. A61K 9/00; A61P 35/00; A61K 39/00

(52) U.S. Cl. CPC A61K 9/0009; A61K 39/0011; A61P 35/00

(57) ABSTRACT

Disclosed are means, methods and compositions of matter useful for amplification of adaptive immune responses towards neoplastic tissue. In one embodiment, immunization of a patient is performed by a means comprising of administering either an exogenous vaccine or stimulation of immunogenicity of the tumor so as to cause release of antigens/increased exposure of antigens, thus resulting in an “endogenous” vaccine. Subsequent to vaccination a patient is treated by an immunopheresis procedure, in order to allow for removal of “blocking factors” produced by the tumor or produced by cells programmed by tumors to produce said blocking factors. In one embodiment further immunization is performed subsequent to removal of said blocking factors in order to allow for enhancement of adaptive immune responses

### CROSS-REFERENCE TO RELATED APPLICATIONS

The present invention claims priority to U.S. Provisional Application No. 62/465,114, filed Feb. 28, 2017, which is hereby incorporated in its entirety including all tables, figures, and claims

### BACKGROUND

The role of the immune system in controlling cancer has been suggested by studies which demonstrate higher incidence of cancer in patients that are chronically immune suppressed. The notion that the immune system plays a role in controlling oncogenesis was described in the 1960s by Burnet, who noticed higher levels of malignancies in patients that suffer from inborn immunodeficiences [1]. This has subsequently been verified over the decades in a variety of neoplasias, and in a variety of immune deficiencies [2]. An example of the impact that congenital immunodeficiency has on incidence of cancer can be seen in a study of 377 patients with primary hypogammaglobulinaemia, mainly common variable immunodeficiency (CVID), 316 patients survived the first 2 years after diagnosis and were the subject of a study of cancer incidence. Among the 220 patients with CVID, there was a 5-fold increase of cancer due mainly to large excesses of stomach cancer (47-fold) and lymphomas (30-fold). The excess of stomach cancer is probably related to the high frequency of achlorhydria in CVID. 3 of the 7 patients with stomach cancer and CVID survived for 5 years or longer [3]. In another study, a 30-fold increase in incidence of colorectal cancer was observed in patients suffering from X-linked agammaglobulinaemia (XLA) [4]. Interestingly, in patients with CVID, the patients that have lower CD8 T cell numbers and activity are more susceptible to cancer development [5].

Immune recognition of cancers is further supported by studies in which infiltration of tumors by lymphocytes is associated with enhanced survival. This has been demonstrated in many tumor types, for example in breast [6], gallbladder [7], and ovarian [8].

The use of immunotherapy for treatment of cancer is well documented. Unfortunately, despite the advent of checkpoint inhibitors, not all patients are responsive to existing therapies. Additionally, checkpoint inhibitors, while inhibiting cancer-induced tolerogenic processes, also have been shown to inhibit natural tolerogenic processes, which in some cases results in autoimmunity. Cases of pathological conditions caused by checkpoint inhibitors include myasthenia gravis [9, 10],

### DESCRIPTION OF THE INVENTION

The invention provides means of using therapeutic “immunopheresis” to amplify potency of adaptive immune responses to cancer and cancer associated antigens. In one embodiment the invention teaches immunization of a cancer patient with a means causes amplification of lymphocyte clones capable of recognizing and inhibiting growth of tumors. Said immunization is followed by an immunopheresis procedure allowing for removal of blocking factors from a patient suffering from cancer. Subsequent to said immunopheresis, additional immunization is performed in order to further expand lymphocytes recognizing tumor associated antigens.

In one embodiment, the invention provides a means for decreasing immunological tolerance, or in some cases breaking immunological tolerance, through the use of immunopheresis. Tolerance may be defined as a selective immunological ignorance of a specific tissue. Numerous examples of tolerance are known in the prior art, for example in pregnancy [11-15], cancer [16-18], transplantation [19, 20], and successfully treated autoimmune conditions [21-24]. In one specific embodiment the invention teaches the use of methods to remove soluble TNF-alpha receptors in a cancer patient. Through the removal of these proteins, the invention teaches, an “anti-tolerogenic” effect is induced, which allows for enhanced ability to immunize against tumor antigens. The reduction of soluble TNF-alpha receptors is performed within the context of the invention for a period of time sufficient to induce immune activation, without inducing chronic inflammation. Indeed, it is known that TNF-alpha produced by the interaction between tumor cells and immune cells, may be assist in tumor growth and other characteristics of cancer such as cachexia. Additionally, it is known that inhibition of TNF-alpha in patients exposed to chronic inflammation, such as patients with rheumatoid arthritis, actually have less cancer incidence, not more [25].

In one embodiment of the invention, immunopheresis is performed to reduce levels of circulating TNF-alpha soluble receptors in order to allow more efficient killing of tumors by radiation, and by combination of radiation and dendritic cell therapy. Indeed, previous studies have shown ability of radiation alone, or combined with dendritic cell therapy to induce increased production of TNF-alpha [26, 27].

In some embodiments of the invention, immunization is performed through induction of localized tumor cell death. In one embodiment, immunopheresis is performed together with isolated limb perfusion of TNF-alpha, means of performing this procedure are known in the art and described in the following references [28-30]. Through inducing local cellular death of neoplastic tissue, resulting endogenous antigens are released which serve as an autologous vaccine [31].

In one embodiment of the invention immunization is performed prior to immunopheresis, with subsequent activation of immunity through reduction of circulating TNF-alpha soluble receptor by immunopheresis. This reduction leads to activation of innate immunity, which serves as the basis for enhancement of tumor immunogenicity. Further enhancement of immunogenicity can be performed by combination with inhibitors of immune inhibitor molecules. Said immune inhibitory molecules are referred to as checkpoint inhibitors and include PD1, PD-L1, CTLA4, TIM3, LAG3, VISTA, BTLA, TIGIT, LAIR1, CD160, 2B4 and TGFR beta. Inhibition of an inhibitory molecule, e.g., by inhibition at the DNA, RNA or protein level, can lead to increased immune function, as described herein. In embodiments, an inhibitory nucleic acid, e.g., an inhibitory nucleic acid, e.g., a dsRNA, e.g., an siRNA or shRNA, can be used to inhibit expression of an inhibitory molecule. In an embodiment the inhibitor is an shRNA. In one embodiment, the inhibitor of an inhibitory signal can be, e.g., an antibody or antibody fragment that binds to an inhibitory molecule. For example, the agent can be an antibody or antibody fragment that binds to PD1, PD-L1, PD-L2 or CTLA4 (e.g., ipilimumab (also referred to as MDX-010 and MDX-101, and marketed as Yervoy®; Bristol-Myers Squibb; Tremelimumab (IgG2 monoclonal antibody available from Pfizer, formerly known as ticilimumab, CP-675,206).). In an embodiment, the agent is an antibody or antibody fragment that binds to TIM3. In an embodiment, the agent is an antibody or antibody fragment that binds to LAG3.

In some embodiments adjuvants to exogenous vaccines are utilized, the adjuvant may be selected from monophosphoryl Lipid A/synthetic trehalose dicorynomycolate (MPL-TDM), AS021/AS02, nonionic block co-polymer adjuvants, CRL 1005, aluminum phosphates, AIPO4), R-848, imiquimod, PAM3CYS, poly (I:C), loxoribine, bacille Calmette-Guerin (BCG), Corynebacterium parvum, CpG oligodeoxynucleotides (ODN), cholera toxin derived antigens, CTA 1-DD, lipopolysaccharide adjuvants, complete Freund's adjuvant, incomplete Freund's adjuvant, saponin, mineral gels, aluminum hydroxide, surface active substances, lysolecithin, pluronic polyols, polyanions, peptides, oil or hydrocarbon emulsions in water, MF59, Montanide ISA 720, keyhole limpet hemocyanins (KLH), dinitrophenol, adenosine receptor inhibitors and combinations thereof.

In some embodiments, the adjuvants are comprised of biodegradable polymeric nanoparticles as delivery vehicles. In some embodiments of all aspects, the biodegradable polymer is selected from the group consisting of a polyester, a lactic acid polymer, copolymers of lactic acid and of glyolic acid (e.g., poly lactic acid (PLA), poly glycolic acid (PGA), or poly (lactic-co-glycolic acid) (“PLGA”), poly-□-caprolactyone (PCL), poly(anhydrides), poly(amides), poly(urethanes), poly(carbonates), poly(acetals), poly(ortho-esters), poly(glycolide-co-trimethylene carbonate), poly(dioxanone), poly(phosphoesters), poly(phosphazenes), poly(cyanoacrylate), poly(ethylene oxide), poly(N-isopropyacrylamide)(PNIPAAm), poly(2-(diethylamine)ethylmethacrylate (PDEAEMA), poly(2-aminoethyl methacrylate) (PAEMA), 2-(dimethylamino)ethyl methacrylate (DMAEMA), poly(ethylene glycol) (PEG), N-(2-hydroxypropyl) methacrylamide (HPMA), poly(□-benzyl-1-aspartate) (PBLA), poly(hydroxybutyrate-co-valerate), derivatives thereof, and mixtures or combinations thereof. In some instances, the copolymers of lactic acid and of glycolic acid are selected from PLA, PGA, and PLGA.

In some aspects the nanoparticles are further comprised of targeting moieties selected from the group consisting of chitosan, mannin, mannitol, polypeptide aptamers, polynucleotide aptamers, RNA aptomers, DNA aptomers, x-aptomers, peptides, polypeptides, antibodies, antibody fragments, Fv fragments, camelids, nanobodies, ligands, RGD, fibronectin and mixtures or combinations thereof.

In some aspects the targets for nanoparticle delivery may be selected from the group consisting of (VCAM)-1, 30.5 kDa antigen, CD34, VEGF, VEGF-VEGFR complex, endosialin, selectins, □v integrins, endoglin, Tie 2, angiostatin receptor, MMP2/MMP9, CD13/Aminopeptidase N, endostatin receptor, TEM1/5/8, VE cadherin cryptic epitope, CD44v3, a,nnexin A1, P-selectin, EDB-Fn, basement membrane component and mixtures or combinations thereof.

As used herein, the term “treatment” refers to clinical intervention in an attempt to alter the natural course of the individual or cell being treated, and may be performed either for prophylaxis or during the course of clinical pathology. Desirable effects include preventing occurrence or recurrence of disease, alleviation of symptoms, and diminishment of any direct or indirect pathological consequences of the disease, preventing metastasis, lowering the rate of disease progression, amelioration or palliation of the disease state, and remission or improved prognosis.

The terms “antigen-presenting cell (s)”, “APC” or “APCs” include both intact, whole cells as well as other molecules (all of allogeneic origin) which are capable of inducing the presentation of one or more antigens, preferably in association with class I MHC molecules, and all types of mononuclear cells which are capable of inducing an allogeneic immune response. Preferably whole viable cells are used as APCs. Examples of suitable APCs are, but not limited to, whole cells such as monocytes, macrophages, DCs, monocyte-derived DCs, macrophage-derived DCs, B cells and myeloid leukemia cells e. g. cell lines THP-1, U937, HL-60 or CEM-CM3. Myeloid leukaemia cells are said to provide so called pre-monocytes.

The terms “cancer”, “neoplasm” and “tumor” are used interchangeably and in either the singular or plural form, as appearing in the present specification and claims, refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but also any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e. g. by such procedures as CAT scan, magnetic resonance imaging (MRI), X-ray, ultrasound or palpation. Non-limiting examples of tumors/cancers relevant for the present invention are carcinomas (e.g. breast cancer, prostate cancer, lung cancer, colorectal cancer, renal cancer, gastric cancer and pancreatic cancer), sarcomas (e.g. bone cancer and synovial cancer), neuro-endocrine tumors (e.g. glioblastoma, medulloblastoma and neuroblastoma), leukemias, lymphomas and squamous cell cancer (e.g. cervical cancer, vaginal cancer and oral cancer). Further, non-limiting examples of tumors/cancers relevant for the present invention are, glioma, fibroblastoma, neurosarcoma, uterine cancer, melanoma, testicular tumors, astrocytoma, ectopic hormone-producing tumor, ovarian cancer, bladder cancer, Wilm's tumor, vasoactive intestinal peptide secreting tumors, head and neck squamous cell cancer, esophageal cancer, or metastatic cancer. Prostate cancer and breast cancer are particularly preferred.

In the context of the present invention the term “culturing” refers to the in vitro propagation of cells or organisms in media of various kinds. It is understood that the descendants of a cell grown in culture may not be completely identical (morphologically, genetically, or phenotypically) to the parent cell. A suitable culturing medium can be selected by the person skilled in the art and examples of such media are RPMI medium or Eagles Minimal Essential Medium (EMEM).

The terms “vaccine”, “immunogen”, or immunogenic composition” are used herein to refer to a compound or composition that is capable of conferring a degree of specific immunity when administered to a human or animal subject. As used in this disclosure, a “cellular vaccine” or “cellular immunogen” refers to a composition comprising at least one cell population, which is optionally inactivated, as an active ingredient. The immunogens, and immunogenic compositions of this invention are active, which mean that they are capable of stimulating a specific immunological response (such as an anti-tumor antigen or anti-cancer cell response) mediated at least in part by the immune system of the host. The immunological response may comprise antibodies, immunoreactive cells (such as helper/inducer or cytotoxic cells), or any combination thereof, and is preferably directed towards an antigen that is present on a tumor towards which the treatment is directed. The response may be elicited or restimulated in a subject by administration of either single or multiple doses.

A compound or composition is “immunogenic” if it is capable of either: a) generating an immune response against an antigen (such as a tumor antigen) in a naive individual; or b) reconstituting, boosting, or maintaining an immune response in an individual beyond what would occur if the compound or composition was not administered. A composition is immunogenic if it is capable of attaining either of these criteria when administered in single or multiple doses.

The term “T-cell response” means the specific proliferation and activation of effector functions induced by a peptide in vitro or in vivo. For MHC class I restricted cytotoxic T cells, effector functions may be lysis of peptide-pulsed, peptide-precursor pulsed or naturally peptide-presenting target cells, secretion of cytokines, preferably Interferon-gamma, TNF-alpha, or IL-2 induced by peptide, secretion of effector molecules, preferably granzymes or perforins induced by peptide, or degranulation.

The term “peptide” is used herein to designate a series of amino acid residues, connected one to the other typically by peptide bonds between the alpha-amino and carbonyl groups of the adjacent amino acids. The peptides are preferably 9 amino acids in length, but can be as short as 8 amino acids in length, and as long as 10, 11, 12, or even longer, and in case of MHC class II peptides (e.g. elongated variants of the peptides of the invention) they can be as long as 15, 16, 17, 18, 19, 20 or 23 or more amino acids in length.

Furthermore, the term “peptide” shall include salts of a series of amino acid residues, connected one to the other typically by peptide bonds between the alpha-amino and carbonyl groups of the adjacent amino acids. Preferably, the salts are pharmaceutical acceptable salts of the peptides, such as, for example, the chloride or acetate (trifluoro-acetate) salts. It has to be noted that the salts of the peptides according to the present invention differ substantially from the peptides in their state(s) in vivo, as the peptides are not salts in vivo.

The term “peptide” shall also include “oligopeptide”. The term “oligopeptide” is used herein to designate a series of amino acid residues, connected one to the other typically by peptide bonds between the alpha-amino and carbonyl groups of the adjacent amino acids. The length of the oligopeptide is not critical to the invention, as long as the correct epitope or epitopes are maintained therein. The oligopeptides are typically less than about 30 amino acid residues in length, and greater than about 15 amino acids in length.

The term “polypeptide” designates a series of amino acid residues, connected one to the other typically by peptide bonds between the alpha-amino and carbonyl groups of the adjacent amino acids. The length of the polypeptide is not critical to the invention as long as the correct epitopes are maintained. In contrast to the terms peptide or oligopeptide, the term polypeptide is meant to refer to molecules containing more than about 30 amino acid residues.

A peptide, oligopeptide, protein or polynucleotide coding for such a molecule is “immunogenic” (and thus is an “immunogen” within the present invention), if it is capable of inducing an immune response. In the case of the present invention, immunogenicity is more specifically defined as the ability to induce a T-cell response. Thus, an “immunogen” would be a molecule that is capable of inducing an immune response, and in the case of the present invention, a molecule capable of inducing a T-cell response. In another aspect, the immunogen can be the peptide, the complex of the peptide with MHC, oligopeptide, and/or protein that is used to raise specific antibodies or TCRs against it.

Cancer-testis antigens: The first TAAs ever identified that can be recognized by T cells belong to this class, which was originally called cancer-testis (CT) antigens because of the expression of its members in histologically different human tumors and, among normal tissues, only in spermatocytes/spermatogonia of testis and, occasion-ally, in placenta. Since the cells of testis do not express class I and II HLA molecules, these antigens cannot be recognized by T cells in normal tissues and can therefore be considered as immunologically tumor-specific. Well-known examples for CT antigens are the MAGE family members and NY-ESO-1. Differentiation antigens: These TAAs are shared between tumors and the normal tissue from which the tumor arose. Most of the known differentiation antigens are found in melanomas and normal melanocytes. Many of these melanocyte lineage-related proteins are involved in biosynthesis of melanin and are therefore not tumor specific but nevertheless are widely used for cancer immunotherapy. Examples include, but are not limited to, tyrosinase and Melan-A/MART-1 for melanoma or PSA for prostate cancer. Over-expressed TAAs: Genes encoding widely expressed TAAs have been detected in histologically different types of tumors as well as in many normal tissues, generally with lower expression levels. It is possible that many of the epitopes processed and potentially presented by normal tissues are below the threshold level for T-cell recognition, while their over-expression in tumor cells can trigger an anticancer response by breaking previously established tolerance. Prominent examples for this class of TAAs are Her-2/neu, survivin, telomerase, or WT1. Tumor-specific antigens: These unique TAAs arise from mutations of normal genes (such as .beta.-catenin, CDK4, etc.). Some of these molecular changes are associated with neoplastic transformation and/or progression. Tumor-specific antigens are generally able to induce strong immune responses without bearing the risk for autoimmune reactions against normal tissues. On the other hand, these TAAs are in most cases only relevant to the exact tumor on which they were identified and are usually not shared between many individual tumors. Tumor-specificity (or -association) of a peptide may also arise if the peptide originates from a tumor-(-associated) exon in case of proteins with tumor-specific (-associated) isoforms. TAAs arising from abnormal post-translational modifications: Such TAAs may arise from proteins which are neither specific nor overexpressed in tumors but nevertheless become tumor associated by posttranslational processes primarily active in tumors. Examples for this class arise from altered glycosylation patterns leading to novel epitopes in tumors as for MUC1 or events like protein splicing during degradation which may or may not be tumor specific. Oncoviral proteins: These TAAs are viral proteins that may play a critical role in the oncogenic process and, because they are foreign (not of human origin), they can evoke a T-cell response. Examples of such proteins are the human papilloma type 16 virus proteins, E6 and E7, which are expressed in cervical carcinoma. T-cell based immunotherapy targets peptide epitopes derived from tumor-associated or tumor-specific proteins, which are presented by molecules of the major histocompatibility complex (MHC). The antigens that are recognized by the tumor specific T lymphocytes, that is, the epitopes thereof, can be molecules derived from all protein classes, such as enzymes, receptors, transcription factors, etc. which are expressed and, as compared to unaltered cells of the same origin, usually up-regulated in cells of the respective tumor.

Therefore, TAAs are a starting point for the development of a T cell based therapy including but not limited to tumor vaccines. The methods for identifying and characterizing the TAAs are usually based on the use of T-cells that can be isolated from patients or healthy subjects, or they are based on the generation of differential transcription profiles or differential peptide expression patterns between tumors and normal tissues. However, the identification of genes over-expressed in tumor tissues or human tumor cell lines, or selectively expressed in such tissues or cell lines, does not provide precise information as to the use of the antigens being transcribed from these genes in an immune therapy. This is because only an individual subpopulation of epitopes of these antigens are suitable for such an application since a T cell with a corresponding TCR has to be present and the immunological tolerance for this particular epitope needs to be absent or minimal. In a very preferred embodiment of the invention it is therefore important to select only those over- or selectively presented peptides against which a functional and/or a proliferating T cell can be found. Such a functional T cell is defined as a T cell, which upon stimulation with a specific antigen can be clonally expanded and is able to execute effector functions (“effector T cell”).

For the purpose of vaccine production, the cancer cells are isolated from an autologous subject, meaning that they will be used to treat the same subject from whom they were derived. Alternatively, the cancer cells could be used in an HLA-matched heterologous subject. Typically the cells are isolated during a biopsy procedure or during surgical tumour removal. The cancer cells may be derived from any type of malignancy and, in an aspect, they are derived from lung cancer, including small cell lung cancer and non-small cell lung cancer (e.g. adenocarcinoma), pancreatic cancer, colon cancer (e.g. colorectal carcinoma, such as, for example, colon adenocarcinoma and colon adenoma), oesophageal cancer, oral squamous carcinoma, tongue carcinoma, gastric carcinoma, liver cancer, nasopharyngeal cancer, hematopoietic tumours of lymphoid lineage (e.g. acute lymphocytic leukemia, B-cell lymphoma, Burkitt's lymphoma), non-Hodgkin's lymphoma (e.g. mantle cell lymphoma), Hodgkin's disease, myeloid leukemia (for example, acute myelogenous leukemia (AML) or chronic myelogenous leukemia (CML)), acute lymphoblastic leukemia, chronic lymphocytic leukemia (CLL), thyroid follicular cancer, myelodysplastic syndrome (MDS), tumours of mesenchymal origin, soft tissue sarcoma, liposarcoma, gastrointestinal stromal sarcoma, malignant peripheral nerve sheath tumour (MPNST), Ewing sarcoma, leiomyosarcoma, mesenchymal chondrosarcoma, lymphosarcoma, fibrosarcoma, rhabdomyosarcoma, melanoma, teratocarcinoma, neuroblastoma, brain tumours, medulloblastoma, glioma, benign tumour of the skin (e.g. keratoacanthoma), breast carcinoma (e.g. advanced breast cancer), kidney carcinoma, nephroblastoma, ovary carcinoma, cervical carcinoma, endometrial carcinoma, bladder carcinoma, prostate cancer, including advanced disease and hormone refractory prostate cancer, testicular cancer, osteosarcoma, head and neck cancer, epidermal carcinoma, multiple myeloma (e.g. refractory multiple myeloma), or mesothelioma. In an aspect, the cancer cells are derived from a solid tumour. Typically, the cancer cells are derived from a breast cancer, colorectal cancer, melanoma, ovarian cancer, pancreatic cancer, gastric cancer, or prostate cancer. More typically, the cancer cells are derived from a prostate cancer. While most cancer cells do not naturally express much if any MHCII on their cell surface, it will be understood that if the cancer cells are derived from antigen-presenting cells, such as a B cell cancer for example, these cells may already express MHCII on their cell surface. It is contemplated that unmodified cancer cells that already express MHCII could be explicitly excluded from the present invention. In other words, it is contemplated that the present invention could encompass cancer cells that are MHCII-negative, MHCII-positive, or both prior to modification according to the present invention. Alternatively, such cells could be included in the invention and it will be understood that, since these cells already express MHCII, incubation with an MHCII-inducing agent is merely optional in order to increase the level of expression. For example, the MHCII-inducing agent may be IFN-.gamma., or it may be an MHCII expression vector that is used to transfect or transduce the cancer cells. The MHCII-inducing agent also encompasses a cell expressing MHCII, in that cells that express MHCII could be fused via cell fusion with the cancer cells to render the cancer cells MHCII positive. Examples of such cells include B cells, dendritic cells, macrophages, and monocytes. In another aspect, the MHCII inducing agent may be an agent that activates the MHCII transactivator (CIITA) sequence. Typically, however, the MHCII-inducing agent is a cytokine, such as, for example, IFN-.alpha., IFN-.beta., IFN-.gamma., IL-4, IL-13, IL-23, or TNF-.alpha. Combinations of cytokines may also be used. In a specific aspect, the MHCII-inducing agent is IFN-.gamma. It is understood that the MHCII-inducing agent may also have effects on increasing expression of MHCI on the cancer cells. For example, if IFN-.gamma. is used as the MHCII-inducing agent, it would also tend to cause an increase in MHCI on the surface of the cancer cells. Once the cancer cells are modified so as to express MHCII, they are incubated with a non-self antigen so that they will present the non-self antigen in the context of MHCII. The non-self antigen can be any antigen that is considered non-self and is capable of inducing an immune response in a subject when presented by MHCII. It will be understood that suitable antigens include antigens that are known to be useful as hapten carriers, such as, for example, thyroglobulin, .beta.-galactosidase, dextran, polylysine, tuberculin derived protein, ovalbumin (OVA), serum albumins such as bovine serum albumin (BSA), sheep serum albumin, goat serum albumins, or fish serum albumin, and keyhole limpet hemocyanin (KLH). The antigen may be derived from the same species as the subject or from a different species. For example, if the subject is a human, the antigen may be a human or non-human antigen. Typically, the antigen is a non-human antigen, such as a bovine, rabbit, murine, canine, or feline antigen, for example. More typically, the antigen is a bovine antigen, such as, for example, bovine serum antigen (BSA). KLH and albumin are other typically used antigens. In an aspect, bovine antigens in general are specifically excluded from the present invention. In another aspect, only BSA is specifically excluded from the present invention. In aspects, the isolated immunogenic cancer cells prepared according to the invention will express both MHCI and MHCII on their cell surface. The MHCI and MHCII molecules will present a number of different antigens as is understood in the art, however, at least some of the MHCI molecules will present tumour-specific antigens and at least some of the MHCII molecules will present non-self antigens. These cells may be then used in an autologous subject for treatment of cancer in the subject. The cells may be used live, attenuated, or killed. Typically, the cells are killed prior to use in a subject by, for example, lethal irradiation, freezing and thawing in the absence of a cryo-preservation agent such as DMSO, or treatment with a cytotoxic compound, such as chemotherapy agents or toxins. If the cells are not for immediate use, they can be preserved, such as, for example, by cryo-preservation, for later administration to the autologous subject. Extracts of the cells may also be used in an autologous subject for treatment of cancer in the subject. For example, the cells may be macerated, sonicated, or otherwise broken up so that they are not in their native whole form. Membrane fractions containing the non-self antigen-bound MHCII molecules may be extracted from the cells and provided in an immunogenic composition for treating cancer in an autologous subject. Additionally, fractions containing just the non-self antigen bound MHCII molecules may be extracted from the cells and provided in an immunogenic composition for treating cancer in an autologous subject. Accordingly, there is therefore provided a cellular extract containing MHCII molecules, wherein the MHCII molecules present a non-self antigen. The extract may further comprise membrane fractions, and it may further comprise MHCI molecules, wherein the MHCI molecules present a cancer antigen. The extract may be provided in an immunogenic composition or a cancer vaccine and may be used to treat an autologous subject with cancer.

The immunogenic compositions and vaccines described herein can be prepared by per se known methods for the preparation of pharmaceutically acceptable compositions that can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, 20th ed., Mack Publishing Company, Easton, Pa., USA, 2000). On this basis, the compositions may include, albeit not exclusively, the cancer cells in association with one or more pharmaceutically acceptable vehicles or diluents, and may be contained in buffered solutions with a suitable pH that are iso-osmotic with physiological fluids. Pharmaceutical compositions include, without limitation, lyophilized powders or aqueous or non-aqueous sterile injectable solutions or suspensions, which may further contain antioxidants, buffers, bacteriostats and solutes that render the compositions substantially compatible with the tissues or the blood of the subject. Other components that may be present in such compositions include water, surfactants (such as Tween), alcohols, polyols, glycerin and vegetable oils, for example. Extemporaneous injection solutions and suspensions may be prepared from sterile powders, granules, tablets, or concentrated solutions or suspensions. The pharmaceutical composition may be supplied, for example, but not by way of limitation, as a lyophilized powder which is reconstituted with sterile water or saline prior to administration to the patient. Suitable pharmaceutically acceptable carriers include essentially chemically inert and nontoxic compositions that do not interfere with the effectiveness of the biological activity of the pharmaceutical composition. Examples of suitable pharmaceutical carriers include, but are not limited to, water, saline solutions, glycerol solutions, ethanol, N-(1(2,3-dioleyloxy)propyl)N,N,N-trimethylammonium chloride (DOTMA), diolesylphosphotidyl-ethanolamine (DOPE), and liposomes. Such compositions should contain a therapeutically effective amount of the modified cancer cells, together with a suitable amount of carrier so as to provide the form for direct administration to the patient.

Any suitable adjuvant may be used in the vaccines of the invention. For example, suitable adjuvants include MPL-TDM adjuvant, AS021/AS02, nonionic block co-polymer adjuvants, aluminum phosphates, R-848, imiquimod, PAM3CYS, poly (I:C), loxoribine, BCG, Corynebacterium parvum, CpG oligodeoxynucleotides, cholera toxin derived antigens, lipopolysaccharide adjuvants, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions in water, keyhole limpet hemocyanins, and dinitrophenol. Typically, the adjuvant used is BCG. The immunogenic compositions and vaccines of the invention may, in aspects, be administered in combination, concurrently or sequentially, with conventional treatments for cancer, including chemotherapy, hormone therapy, biotherapy, and radiation therapy, for example. The compositions of the invention may be formulated together with such conventional treatments when appropriate.

In some embodiments of the invention induction of antigen release is accomplished by administration of a “Chemotherapeutic agent” or combination of said agents, these include chemical compounds useful in the treatment of cancer. Examples of chemotherapeutic agents include erlotinib (TARCEVA®, Genentech/OSI Pharm.), bortezomib (VELCADE®, Millennium Pharm.), disulfiram, epigallocatechin gallate, salinosporamide A, carfilzomib, 17-AAG (geldanamycin), radicicol, lactate dehydrogenase A (LDH-A), fulvestrant (FASLODEX®, AstraZeneca), sunitib (SUTENT®, Pfizer/Sugen), letrozole (FEMARA®, Novartis), imatinib mesylate (GLEEVEC®, Novartis), finasunate (VATALANIB®, Novartis), oxaliplatin (ELOXATIN®, Sanofi), 5-FU (5-fluorouracil), leucovorin, Rapamycin (Sirolimus, RAPAMUNE®, Wyeth), Lapatinib (TYKERB®, GSK572016, Glaxo Smith Kline), Lonafamib (SCH 66336), sorafenib (NEXAVAR®, Bayer Labs), gefitinib (IRESSA®, AstraZeneca), AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5.alpha.-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin .gamma.1I and calicheamicin .omega.1I (Angew Chem. Intl. Ed. Engl. 1994 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (docetaxel, doxetaxel; Sanofi-Aventis); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above. Chemotherapeutic agent also includes (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN®, rIL-2; a topoisomerase 1 inhibitor such as LURTOTECAN®; ABARELIX® rmRH; and (ix) pharmaceutically acceptable salts, acids and derivatives of any of the above. Chemotherapeutic agent also includes antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds of the invention include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories) which is a recombinant exclusively human-sequence, full-length IgG1.lamda. antibody genetically modified to recognize interleukin-12 p40 protein. Chemotherapeutic agent also includes “EGFR inhibitors,” which refers to compounds that bind to or otherwise interact directly with EGFR and prevent or reduce its signaling activity, and is alternatively referred to as an “EGFR antagonist.” Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBUTIX®) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding (EMD/Merck); human EGFR antibody, HuMax-EGFR (GenMab); fully human antibodies known as E1.1, E2.4, E2.5, E6.2, E6.4, E2.11, E6.3 and E7.6.3 and described in U.S. Pat. No. 6,235,883; MDX-447 (Medarex Inc); and mAb 806 or humanized mAb 806 (Johns et al., J. Biol. Chem. 279(29):30375-30384 (2004)). The anti-EGFR antibody may be conjugated with a cytotoxic agent, thus generating an immunoconjugate (see, e.g., EP659,439A2, Merck Patent GmbH). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFR antagonists include OSI-774 (CP-358774, erlotinib, TARCEVA® Genentech/OSI Pharmaceuticals); PD 183805 (CI 1033, 2-propenamide, N-[4-[(3-chloro-4-fluorophenyl)amino]-7-[3-(4-morpholinyl)propoxy]-6-quin-azolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoli-ne, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol)-; (R)-6-(4-hydroxyphenyl)-4-[(1-phenylethyl)amino]-7H-pyrrolo[2,3-d]pyrimi-dine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(-dimethylamino)-2-butenamide) (Wyeth); AG1478 (Pfizer); AG1571 (SU 5271; Pfizer); dual EGFR/HER2 tyrosine kinase inhibitors such as lapatinib (TYKERB®, GSK572016 or N-[3-chloro-4-[(3 fluorophenyl)methoxy]phenyl]-6[5[[[2methylsulfonyl)ethyl]amino]methyl]-2-furanyl]-4-quinazolinamine). Chemotherapeutic agents also include “tyrosine kinase inhibitors” including the EGFR-targeted drugs noted in the preceding paragraph; small molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibit Raf-1 signaling; non-HER targeted TK inhibitors such as imatinib mesylate (GLEEVEC®, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase I inhibitor CI-1040 (available from Pharmacia); quinazolines, such as PD 153035, 4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d]pyrimidines; curcumin (diferuloyl methane, 4,5-bis(4-fluoroanilino)phthalimide); tyrphostines containing nitrothiophene moieties; PD-0183805 (Warner-Lamber); antisense molecules (e.g. those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); imatinib mesylate (GLEEVEC®); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone), rapamycin (sirolimus, RAPAMUNE®); or as described in any of the following patent publications: U.S. Pat. No. 5,804,396; WO 1999/09016 (American Cyanamid); WO 1998/43960 (American Cyanamid); WO 1997/38983 (Warner Lambert); WO 1999/06378 (Warner Lambert); WO 1999/06396 (Warner Lambert); WO 1996/30347 (Pfizer, Inc); WO 1996/33978 (Zeneca); WO 1996/3397 (Zeneca) and WO 1996/33980 (Zeneca). Chemotherapeutic agents also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof. Chemotherapeutic agents also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate and fluprednidene acetate; immune selective anti-inflammatory peptides (ImSAIDs) such as phenylalanine-glutamine-glycine (FEG) and its D-isomeric form (feG) (IMULAN BioTherapeutics, LLC); anti-rheumatic drugs such as azathioprine, ciclosporin (cyclosporine A), D-penicillamine, gold salts, hydroxychloroquine, leflunomideminocycline, sulfasalazine, tumor necrosis factor alpha (TNF.alpha.) blockers such as etanercept (Enbrel), infliximab (Remicade), adalimumab (Humira), certolizumab pegol (Cimzia), golimumab (Simponi), Interleukin 1 (IL-1) blockers such as anakinra (Kineret), T cell costimulation blockers such as abatacept (Orencia), Interleukin 6 (IL-6) blockers such as tocilizumab (ACTEMERA®); Interleukin 13 (IL-13) blockers such as lebrikizumab; Interferon alpha (IFN) blockers such as Rontalizumab; Beta 7 integrin blockers such as rhuMAb Beta7; IgE pathway blockers such as Anti-M1 prime; Secreted homotrimeric LTa3 and membrane bound heterotrimer LTa1/.beta.2 blockers such as Anti-lymphotoxin alpha (LTa); radioactive isotopes (e.g., At211, I131, I125, Y90, Re186, Re188, Sm153, Bi212, P32, Pb212 and radioactive isotopes of Lu); miscellaneous investigational agents such as thioplatin, PS-341, phenylbutyrate, ET-18-OCH3, or farnesyl transferase inhibitors (L-739749, L-744832); polyphenols such as quercetin, resveratrol, piceatannol, epigallocatechine gallate, theaflavins, flavanols, procyanidins, betulinic acid and derivatives thereof; autophagy inhibitors such as chloroquine; delta-9-tetrahydrocannabinol (dronabinol, MARINOL®); beta-lapachone; lapachol; colchicines; betulinic acid; acetylcamptothecin, scopolectin, and 9-aminocamptothecin); podophyllotoxin; tegafur (UFTORAL®); bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); and epidermal growth factor receptor (EGF-R); vaccines such as THERATOPE® vaccine; perifosine, COX-2 inhibitor (e.g. celecoxib or etoricoxib), proteosome inhibitor (e.g. PS341); CCI-779; tipifarnib (R11577); orafenib, ABT510; Bc1-2 inhibitor such as oblimersen sodium (GENASENSE®); pixantrone; farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin. Chemotherapeutic agents also include non-steroidal anti-inflammatory drugswith analgesic, antipyretic and anti-inflammatory effects. NSAIDs include non-selective inhibitors of the enzyme cyclooxygenase. Specific examples of NSAIDs include aspirin, propionic acid derivatives such as ibuprofen, fenoprofen, ketoprofen, flurbiprofen, oxaprozin and naproxen, acetic acid derivatives such as indomethacin, sulindac, etodolac, diclofenac, enolic acid derivatives such as piroxicam, meloxicam, tenoxicam, droxicam, lornoxicam and isoxicam, fenamic acid derivatives such as mefenamic acid, meclofenamic acid, flufenamic acid, tolfenamic acid, and COX-2 inhibitors such as celecoxib, etoricoxib, lumiracoxib, parecoxib, rofecoxib, rofecoxib, and valdecoxib. NSAIDs can be indicated for the symptomatic relief of conditions such as rheumatoid arthritis, osteoarthritis, inflammatory arthropathies, ankylosing spondylitis, psoriatic arthritis, Reiter's syndrome, acute gout, dysmenorrhoea, metastatic bone pain, headache and migraine, postoperative pain, mild-to-moderate pain due to inflammation and tissue injury, pyrexia, ileus, and renal colic.

In an embodiment the anti-cancer vaccine is produced by a method described herein. In an embodiment the anti-cancer vaccine comprises solubilized and reduced components of cancer cells or cancer-associated cells. In an embodiment the anti-cancer vaccine comprises solubilized and reduced components of cancer cells or cancer-associated cells, and a non-mammalian polypeptide capable of binding a mammalian protein. In an embodiment the anti-cancer vaccine comprises solubilized, reduced and alkylated components of cancer cells or cancer-associated cells, and a non-mammalian polypeptide capable of binding a mammalian protein. In an embodiment the non-mammalian polypeptide is streptavidin, avidin or Neutravidin. In a particular preferred embodiment the non-mammalian polypeptide is streptavidin. In an embodiment the cancer cells or cancer-associated cells are from the subject intended to receive the composition. In an embodiment the anti-cancer vaccine is produced by a method described herein. In an embodiment the anti-cancer vaccine comprises solubilized and reduced components of cancer cells or cancer-associated cells. In an embodiment the anti-cancer vaccine comprising solubilized and reduced components of cancer cells or cancer-associated cells, further comprises a non-mammalian polypeptide capable of binding a mammalian protein. In an embodiment the anti-cancer vaccine comprises solubilized, reduced and alkylated components of cancer cells or cancer-associated cells, and a non-mammalian polypeptide capable of binding a mammalian protein. In an embodiment the cancer cells or cancer-associated cells are from the subject intended to receive the vaccine and the composition. In an embodiment administration is at or near the site of the tumour. In an embodiment administration is remote from the site of the tumor. The biological sample may be any sample from a subject which includes at least one cancer cell or cancer-associated cell, including, but not limited to tissue, tissue fluids, blood, blood components, bone marrow, excreta including urine and feces, and secreta including mucus. The biological sample may be more than one type. For example, a biological sample may be comprised of a tissue sample and a blood sample. The biological sample may comprise a tissue sample from one site on a subject and a tissue sample from another site on a subject. The biological sample may comprise more than one sample taken from a subject at different times. For example, a biological sample may comprise two blood samples that are taken from a subject on two separate occasions. In a preferred embodiment, the biological sample comprises a biopsy of a known or suspected cancer or tumour. The biological sample comprising at least one cancer cell or cancer-associated cell may, for example, be a tumour sample. The biological sample will typically comprise cancer cells and non-cancer cells, and non-cellular components such as, for example, plasma, extra-cellular matrix, enzymes, growth factors and cytokines. The biological sample may be collected from a subject under the clinical care of a medical practitioner by, for example, a medical practitioner or a health care professional. A medical practitioner may be any person that is registered, authorized or certified under law to practice medicine independently. A health care professional may be any person that is permitted, authorized, registered or certified to collect a biological sample from a subject either independently or under the supervision of a medical practitioner. For example, the health care professional may be a registered or enrolled nurse, or a medical practitioner's assistant or a clinical assistant. It would be understood that the biological sample may, for example, be collected during routine out-patient procedures that would ordinarily be carried out on a subject with cancer who is under the clinical care of a medical practitioner. In a particular embodiment, the method of the present invention is performed by a medical practitioner or by a person or persons under the supervision of a medical practitioner, or by a combination thereof. A person under the supervision of a medical practitioner may be, for example, a health care professional, a pharmacist, a clinical, medical or pathology laboratory technician, or a scientist. It would be understood that the method of the present invention may be performed in any laboratory by a medical practitioner or by a person or persons under the supervision of a medical practitioner, or by a combination thereof.

Various aspects of the invention of the invention relating to the above are enumerated in the following paragraphs:

Aspect 1. A method of augmenting an existing antigen-specific immune response towards a neoplastic cell comprising the steps of: a) selecting a patient suffering from a cancer; b) identifying existing immune responses towards the cancer; c) performing one or more immunopheresis procedures subsequent to vaccinating towards antigens associated with existing antigen-specific immune response; d) optionally performing vaccination towards antigens associated with said existing antigen-specific immune response subsequent to one or more immunopheresis procedures.

Aspect 2. The method of aspect 1, wherein said cancer is a condition in which cells of the body possess one or more properties selected from a group comprising of: a) uncontrolled growth; b) apoptosis resistance; c) metastasis; d) immune suppression; and e) cellular immortality.

Aspect 3. The method of aspect 1, wherein said cancers are selected from a group comprising of: brain, melanoma, bladder, breast, cervix, colon, head and neck, kidney, lung, non-small cell lung, mesothelioma, ovary, prostate, sarcoma, stomach, uterus, as well as carcinomas, including pindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrmcous carcinoma, carcinoma villosum, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, and carcinoma scroti, The term “sarcoma” generally refers to a tumor which is made up of a substance like the embryonic connective tissue and is generally composed of closely packed cells embedded in a fibrillar, heterogeneous, or homogeneous substance. Sarcomas include, chondrosarcoma, fibrosarcoma, lymphosarcoma, melanosarcoma, myxosarcoma, osteosarcoma, endometrial sarcoma, stromal sarcoma, Ewing's sarcoma, fascial sarcoma, fibroblastic sarcoma, giant cell sarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilns' tumor sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

Aspect 4. The method of aspect 1, wherein said existing immune responses to said cancer are identified by selection of antibodies recognizing tumor associated antigens.

Aspect 5. The method of aspect 1, wherein said existing immune responses to said cancer are identified by selection of T cells recognizing tumor associated antigens.

Aspect 6. The method of aspect 5, wherein said T cells are capable of coordinating immune responses against said tumor.

Aspect 7. The method of aspect 4, wherein said antibodies recognizing said tumor antigens are selected for by an enzyme linked immunosorbent assay (ELISA), wherein said ELISA utilizes bound tumor antigens to screen for antibodies obtained from said cancer patients, wherein said antibodies recognize preselected tumor antigens.

Aspect 8. The method of aspect 7, wherein said tumor associated antigens are derived from said patient's own tumor.

Aspect 9. The method of aspect 7, wherein said tumor associated antigens are derived from said patient's own tumor by means of a biopsy.

Aspect 10. The method of aspect 7, wherein said tumor associated antigens are derived from said patient's own tumor by means of selecting circulating tumor cells.

Aspect 11. The method of aspect 7, wherein said tumor associated antigens are derived from said patient's own tumor by means of selecting tumor derived microparticles in circulation.

Aspect 12. The method of aspect 7, wherein said tumor associated antigens are proteins selected from a group comprising of: a) Fos-related antigen 1; b) LCK; c) FAP; d) VEGFR2; e) NA17; f) PDGFR-beta; g) PAP; h) MAD-CT-2; i) Tie-2; j) PSA; k) protamine 2; l) legumain; m) endosialin; n) prostate stem cell antigen; o)carbonic anhydrase IX; p) STn; q) Page4; r) proteinase 3; s) GM3 ganglioside; t) tyrosinase; u) MART1; v) gp100; w) SART3; x) RGS5; y) SSX2; z) Globol1; aa) Tn; ab) CEA; ac) hCG; ad) PRAME; ae) XAGE-1; af) AKAP-4; ag) TRP-2; ah) B7H3; ai) sperm fibrous sheath protein; aj) CYP1B1; ak) HMWMAA; al) sLe(a); am) MAGE A1; an) GD2; ao) PSMA; ap) mesothelin; aq) fucosyl GM1; ar) GD3; as) sperm protein 17; at) NY-ESO-1; au) PAX5; av) AFP; aw) polysialic acid; ax) EpCAM; ay) MAGE-A3; az) mutant p53; ba) ras; bb) mutant ras; bc) NY-BR1; bd) PAX3; be) HER2/neu; bf) OY-TES1; bg) HPV E6 E7; bh) PLAC1; bi) hTERT; bj) BORIS; bk) ML-IAP; bl) idiotype of b cell lymphoma or multiple myeloma; bm) EphA2; bn) EGFRvIII; bo) cyclin B1; bp) RhoC; bq) androgen receptor; br) surviving; bs) MYCN; bt) wildtype p53; bu) LMP2; by) ETV6-AML; bw) MUC1; bx) BCR-ABL; by) ALK; bz) WT1; ca) ERG (TMPRSS2 ETS fusion gene); cb) sarcoma translocation breakpoint; cc) STEAP; cd) OFA/iLRP; and ce) Chondroitin sulfate proteoglycan 4 (CSPG4).

Aspect 13. The method of aspect 12, wherein said tumor associated antigens are immunogenic peptides derived from proteins selected from a proteins comprising of: a) Fos-related antigen 1; b) LCK; c) FAP; d) VEGFR2; e) NA17; f) PDGFR-beta; g) PAP; h) MAD-CT-2; i) Tie-2; j) PSA; k) protamine 2; l) legumain; m) endosialin; n) prostate stem cell antigen; o)carbonic anhydrase IX; p) STn; q) Page4; r) proteinase 3; s) GM3 ganglioside; t) tyrosinase; u) MART1; v) gp100; w) SART3; x) RGS5; y) SSX2; z) Globol1; aa) Tn; ab) CEA; ac) hCG; ad) PRAME; ae) XAGE-1; af) AKAP-4; ag) TRP-2; ah) B7H3; ai) sperm fibrous sheath protein; aj) CYP1B1; ak) HMWMAA; al) sLe(a); am) MAGE A1; an) GD2; ao) PSMA; ap) mesothelin; aq) fucosyl GM1; ar) GD3; as) sperm protein 17; at) NY-ESO-1; au) PAX5; av) AFP; aw) polysialic acid; ax) EpCAM; ay) MAGE-A3; az) mutant p53; ba) ras; bb) mutant ras; bc) NY-BR1; bd) PAX3; be) HER2/neu; bf) OY-TES1; bg) HPV E6 E7; bh) PLAC1; bi) hTERT; bj) BORIS; bk) ML-IAP; bl) idiotype of b cell lymphoma or multiple myeloma; bm) EphA2; bn) EGFRvIII; bo) cyclin B1; bp) RhoC; bq) androgen receptor; br) surviving; bs) MYCN; bt) wildtype p53; bu) LMP2; by) ETV6-AML; bw) MUC1; bx) BCR-ABL; by) ALK; bz) WT1; ca) ERG (TMPRSS2 ETS fusion gene); cb) sarcoma translocation breakpoint; cc) STEAP; cd) OFA/iLRP; and ce) Chondroitin sulfate proteoglycan 4 (CSPG4).

Aspect 14. The method of aspect 4, wherein subsequent to identification of one or more antigens which are reactive to antibodies found in a patient suffering from cancer, said antigens are used for generation of a therapeutic vaccine.

Aspect 15. The method of aspect 5, wherein T cells reactive to tumor antigens are identified by use of one or more major histocompatibility complex proteins (MHC) loaded with tumor antigens or peptides derived thereof.

Aspect 16. The method of aspect 15, wherein said MHC protein is MHC class I.

Aspect 17. The method of aspect 15, wherein said MHC protein is MHC class II.

Aspect 18. The method of aspect 5, wherein T cell reactivity is assessed by proliferation of T cells in response to presentation of antigen together with a costimulatory molecule.

Aspect 19. The method of aspect 18, wherein said costimulatory molecule is provided as an antigen presenting cell.

Aspect 20. The method of aspect 19, wherein said antigen presenting cell is a dendritic cell.

Aspect 21. The method of aspect 20, wherein said dendritic cell expresses CD83.

Aspect 22. The method of aspect 20, wherein said dendritic cell expresses CD80.

Aspect 23. The method of aspect 20, wherein said dendritic cell expresses CD86.

Aspect 24. The method of aspect 20, wherein said dendritic cell produces IL-12.

Aspect 25. The method of aspect 19, wherein said antigen presenting cell is a B cell.

Aspect 26. The method of aspect 19, wherein said antigen presenting cell is an artificial antigen presenting cell.

Aspect 27. The method of aspect 26, wherein said artificial antigen presenting cell is generated by transfecting a non-antigen presenting cell with molecules capable of endowing antigen presentation.

Aspect 28. The method of aspect 27, wherein said molecules capable of endowing antigen presentation include: a) MHC I; b) MHC II; c) CD80; d) CD86; e) IL-12; f) CLIP; g) IL-2 and h) CD40.

Aspect 29. The method of aspect 5, wherein T cell reactivity is assessed by production of cytokines by T cells in response to presentation of antigen together with a costimulatory molecule.

Aspect 30. The method of aspect 29, wherein said cytokine produced as a consequence to T cell activation subsequent to binding of tumor antigen is IL-2.

Aspect 31. The method of aspect 29, wherein said cytokine produced as a consequence to T cell activation subsequent to binding of tumor antigen is IL-7.

Aspect 32. The method of aspect 29, wherein said cytokine produced as a consequence to T cell activation subsequent to binding of tumor antigen is IL-12.

Aspect 33. The method of aspect 29, wherein said cytokine produced as a consequence to T cell activation subsequent to binding of tumor antigen is IL-17.

Aspect 34. The method of aspect 29, wherein said cytokine produced as a consequence to T cell activation subsequent to binding of tumor antigen is IL-18.

Aspect 35. The method of aspect 5, wherein said T cell reactivity is assessed by induction of expression of IL-2 receptor.

Aspect 36. The method of aspect 36, wherein said IL-2 receptor is CD25.

Aspect 37. The method of aspect 5, wherein said T cell reactivity is assessed by induction of expression of CD69.

Aspect 38. The method of aspect 5, wherein said T cell reactivity is assessed by induction of expression of 4.1-BB.

Aspect 39. The method of aspect 5, wherein said tumor associated antigens are derived from said patient's own tumor.

Aspect 40. The method of aspect 5, wherein said tumor associated antigens are derived from said patient's own tumor by means of a biopsy.

Aspect 41. The method of aspect 5, wherein said tumor associated antigens are derived from said patient's own tumor by means of selecting circulating tumor cells.

Aspect 42. The method of aspect 5, wherein said tumor associated antigens are derived from said patient's own tumor by means of selecting tumor derived microparticles in circulation.

Aspect 43. The method of aspect 5, wherein said tumor associated antigens are immunogenic peptides derived from proteins, said proteins selected from a group comprising of: a) Fos-related antigen 1; b) LCK; c) FAP; d) VEGFR2; e) NA17; f) PDGFR-beta; g) PAP; h) MAD-CT-2; i) Tie-2; j) PSA; k) protamine 2; l) legumain; m) endosialin; n) prostate stem cell antigen; o)carbonic anhydrase IX; p) STn; q) Page4; r) proteinase 3; s) GM3 ganglioside; t) tyrosinase; u) MART1; v) gp100; w) SART3; x) RGS5; y) SSX2; z) Globol1; aa) Tn; ab) CEA; ac) hCG; ad) PRAME; ae) XAGE-1; af) AKAP-4; ag) TRP-2; ah) B7H3; ai) sperm fibrous sheath protein; aj) CYP1B1; ak) HMWMAA; al) sLe(a); am) MAGE A1; an) GD2; ao) PSMA; ap) mesothelin; aq) fucosyl GM1; ar) GD3; as) sperm protein 17; at) NY-ESO-1; au) PAX5; av) AFP; aw) polysialic acid; ax) EpCAM; ay) MAGE-A3; az) mutant p53; ba) ras; bb) mutant ras; bc) NY-BR1; bd) PAX3; be) HER2/neu; bf) OY-TES1; bg) HPV E6 E7; bh) PLAC1; bi) hTERT; bj) BORIS; bk) ML-IAP; bl) idiotype of b cell lymphoma or multiple myeloma; bm) EphA2; bn) EGFRvIII; bo) cyclin B1; bp) RhoC; bq) androgen receptor; br) surviving; bs) MYCN; bt) wildtype p53; bu) LMP2; by) ETV6-AML; bw) MUC1; bx) BCR-ABL; by) ALK; bz) WT1; ca) ERG (TMPRSS2 ETS fusion gene); cb) sarcoma translocation breakpoint; cc) STEAP; cd) OFA/iLRP; and ce) Chondroitin sulfate proteoglycan 4 (CSPG4).

Aspect 44. The method of aspect 5, wherein subsequent to identification of one or more antigens which are capable of stimulating T cells found in a patient suffering from cancer, said antigens are used for generation of a therapeutic vaccine.

Aspect 45. The method of aspect 1, wherein said vaccine generated to amplify existing immune response in a cancer patient is admixed with an adjuvant.

Aspect 46. The method of aspect 45, wherein said adjuvant is capable of stimulating maturation of dendritic cells.

Aspect 47. The method of aspect 45, wherein said adjuvant is a dendritic cell which has been pulsed with a tumor antigen ex vivo.

Aspect 48. The method of aspect 47, wherein said ex vivo generated dendritic cell is autologous to the patient in need of therapy.

Aspect 49. The method of aspect 47, wherein said ex vivo generated dendritic cell is allogeneic to the patient in need of therapy.

Aspect 50. The method of aspect 47, wherein said ex vivo generated dendritic cell is generated by culture of dendritic cell progenitors in a combination of interleukin-4 and GM-CSF at a concentration and duration sufficient for differentiation into dendritic cells.

Aspect 51. The method of aspect 50, wherein said dendritic cell progenitors are monocytes.

Aspect 52. The method of aspect 50, wherein said dendritic cell progenitors are obtained from cord blood.

Aspect 53. The method of aspect 50, wherein said dendritic cell progenitors are obtained from peripheral blood.

Aspect 54. The method of aspect 50, wherein said dendritic cell progenitors are obtained from bone marrow.

Aspect 55. The method of aspect 50, wherein said dendritic cell progenitors are obtained from mobilized peripheral blood.

Aspect 56. The method of aspect 55, wherein said mobilization of peripheral blood is accomplished by administration of G-CSF.

Aspect 57. The method of aspect 55, wherein said mobilization of peripheral blood is accomplished by administration of an antagonist to CXCR4.

Aspect 58. The method of aspect 55, wherein said antagonist to CXCR4 is Mozibil.

Aspect 59. The method of aspect 46, wherein said adjuvant is an agent or plurality of agents capable of inducing in vivo dendritic cell maturation.

Aspect 60. The method of aspect 59, wherein said adjuvant capable of inducing in vivo dendritic cell maturation is an activator of a toll like receptor.

Aspect 61. The method of aspect 60, wherein said toll like receptor is TLR-1.

Aspect 62. The method of aspect 61, wherein said activator of TLR-1 is Pam3CSK4.

Aspect 63. The method of aspect 60, wherein said toll like receptor is TLR-2.

Aspect 64. The method of aspect 63, wherein said activator of TLR-2 is HKLM.

Aspect 65. The method of aspect 60, wherein said toll like receptor is TLR-3.

Aspect 66. The method of aspect 65, wherein said activator of TLR-3 is Poly:IC.

Aspect 67. The method of aspect 60, wherein said toll like receptor is TLR-4.

Aspect 68. The method of aspect 67, wherein said activator of TLR-4 is LPS.

Aspect 69. The method of aspect 67, wherein said activator of TLR-4 is Buprenorphine.

Aspect 70. The method of aspect 67, wherein said activator of TLR-4 is Carbamazepine.

Aspect 71. The method of aspect 67, wherein said activator of TLR-4 is Fentanyl.

Aspect 72. The method of aspect 67, wherein said activator of TLR-4 is Levorphanol.

Aspect 73. The method of aspect 67, wherein said activator of TLR-4 is Methadone.

Aspect 74. The method of aspect 67, wherein said activator of TLR-4 is Morphine.

Aspect 75. The method of aspect 67, wherein said activator of TLR-4 is Oxcarbazepine.

Aspect 76. The method of aspect 67, wherein said activator of TLR-4 is Oxycodone.

Aspect 77. The method of aspect 67, wherein said activator of TLR-4 is Pethidine.

Aspect 78. The method of aspect 67, wherein said activator of TLR-4 is Glucuronoxylomannan from Cryptococcus.

Aspect 79. The method of aspect 67, wherein said activator of TLR-4 is Morphine-3-glucuronide.

Aspect 80. The method of aspect 67, wherein said activator of TLR-4 is lipoteichoic acid.

Aspect 81. The method of aspect 67, wherein said activator of TLR-4 is β-defensin 2.

Aspect 82. The method of aspect 67, wherein said activator of TLR-4 is small molecular weight hyaluronic acid.

Aspect 83. The method of aspect 67, wherein said activator of TLR-4 is fibronectin EDA.

Aspect 84. The method of aspect 67, wherein said activator of TLR-4 is snapin.

Aspect 85. The method of aspect 67, wherein said activator of TLR-4 is tenascin C.

Aspect 86. The method of aspect 60, wherein said toll like receptor is TLR-5.

Aspect 87. The method of aspect 86, wherein said activator of TLR-5 is flagellin.

Aspect 88. The method of aspect 60, wherein said toll like receptor is TLR-6.

Aspect 89. The method of aspect 67, wherein said activator of TLR-6 is FSL-1.

Aspect 90. The method of aspect 60, wherein said toll like receptor is TLR-7.

Aspect 91. The method of aspect 90, wherein said activator of TLR-7 is imiquimod.

Aspect 92. The method of aspect 60, wherein said toll like receptor of TLR-8.

Aspect 93. The method of aspect 92, wherein said activator of TLR8 is ssRNA40/LyoVec.

Aspect 94. The method of aspect 60, wherein said toll like receptor of TLR-9.

Aspect 95. The method of aspect 94, wherein said activator of TLR-9 is a CpG oligonucleotide.

Aspect 96. The method of aspect 94, wherein said activator of TLR-9 is ODN2006.

Aspect 97. The method of aspect 94, wherein said activator of TLR-9 is Agatolimod.

Aspect 98. The method of aspect 45, wherein said adjuvant is administered together with an inhibitor of an immune suppressive molecule or molecules.

Aspect 99. The method of aspect 98, wherein said inhibitor of said immune suppressive molecule is an antibody to interleukin-10.

Aspect 100. The method of aspect 98, wherein said inhibitor of said immune suppressive molecule is an antibody to interleukin-4.

Aspect 101. The method of aspect 98, wherein said inhibitor of said immune suppressive molecule is an antibody to VEGF.

Aspect 102. The method of aspect 98, wherein said inhibitor of said immune suppressive molecule is an antibody to interleukin-13.

Aspect 103. The method of aspect 98, wherein said inhibitor of said immune suppressive molecule is an antibody to interleukin-20.

Aspect 104. The method of aspect 98, wherein said inhibitor of said immune suppressive molecule is an inhibitor of PGE-2 production.

Aspect 105. The method of aspect 104, wherein said inhibitor of PGE-2 production is etoricoxib.

Aspect 106. The method of aspect 104, wherein said inhibitor of PGE-2 production is celecoxib.

Aspect 107. The method of aspect 104, wherein said inhibitor of PGE-2 production is rofecoxib.

Aspect 108. The method of aspect 104, wherein said inhibitor of PGE-2 production is valdecoxib.

Aspect 109. The method of aspect 104, wherein said inhibitor of PGE-2 production is one or more omega-3 fatty acids.

Aspect 110. The method of aspect 104, wherein said inhibitor of PGE-2 production is white willow bark.

Aspect 111. The method of aspect 104, wherein said inhibitor of PGE-2 production is curcumin.

Aspect 112. The method of aspect 104, wherein said inhibitor of PGE-2 production is epigallocatechin-3 galate.

Aspect 113. The method of aspect 104, wherein said inhibitor of PGE-2 production is pycnogenol.

Aspect 114. The method of aspect 104, wherein said inhibitor of PGE-2 production is boswellia serrata resin.

Aspect 115. The method of aspect 98, wherein said immune suppressive molecule is a purinergic receptor.

Aspect 116. The method of aspect 98, wherein said immune suppressive molecule is CD39.

Aspect 117. The method of aspect 98, wherein said immune suppressive molecule is CD73.

Aspect 118. The method of aspect 98, wherein said immune suppressive molecule is NR2F6.

Aspect 119. The method of aspect 98, wherein said immune suppressive molecule is an immunological checkpoint.

Aspect 120. The method of aspect 119, wherein said immunological checkpoint is one or more selected from a group comprising of: a) CTLA-4; b) PD-1; c) PD-1 ligand; d) TIM-3; and e) BTLA-4.

Aspect 121. The method of aspect 1, wherein said immunopheresis procedure is performed to extract immune suppressive entities from circulation of a patient in need of treatment.

Aspect 122. The method of aspect 121, wherein said immune suppressive entities are cytokines.

Aspect 123. The method of aspect 122, wherein said cytokine is interleukin-10.

Aspect 124. The method of aspect 122, wherein said cytokine is VEGF.

Aspect 125. The method of aspect 122, wherein said cytokine is TGF-beta.

Aspect 126. The method of aspect 122, wherein said cytokine is osteopontin.

Aspect 127. The method of aspect 121, wherein said immunosuppressive entity is a soluble protein.

Aspect 128. The method of aspect 127, wherein said soluble protein is soluble interleukin-2 receptor.

Aspect 129. The method of aspect 127, wherein said soluble protein is soluble tumor necrosis factor alpha receptor.

Aspect 130. The method of aspect 127, wherein said soluble protein is soluble Fas ligand.

Aspect 131. The method of aspect 127, wherein said soluble protein is soluble HLA-G.

Aspect 132. The method of aspect 127, wherein said soluble protein is soluble MICA.

Aspect 133. The method of aspect 127, wherein said soluble protein is interleukin-1 receptor antagonist.

Aspect 134. The method of aspect 121, wherein said immune suppressive entities are tumor secreted exosomes.

Aspect 135. The method of aspect 134, wherein said tumor secreted exosomes express higher concentration of Fas ligand as compared to exosomes found in non-tumor bearing patients.

Aspect 136. The method of aspect 134, wherein said tumor secreted exosomes express higher concentration of glycosylated proteins as compared to exosomes found in non-tumor bearing patients.

Aspect 137. The method of aspect 134, wherein said tumor secreted exosomes express higher concentration of EGF-R as compared to exosomes found in non-tumor bearing patients.

Aspect 138. The method of aspect 134, wherein said tumor secreted exosomes express higher concentration of CSPG4 as compared to exosomes found in non-tumor bearing patients.

Aspect 139. A method of augmenting immunity to a specific tumor antigen, wherein a patient is administered a tumor antigen representing an existing tumor, said administration performed in an immunogenic manner, followed by immunopheresis procedure.

Aspect 140. The method of aspect 139, wherein administration of said tumor antigen in said immunogenic manner is performed subsequent to said immunopheresis procedure.

Aspect 141. The method of aspect 140, wherein said antigen is selected from a group of antigens comprising of: AMACR, PAP, PSM, MAGE, NY-ESO-1, MUM-1, p53, CDK4, HER2/NEU, antigens from Papilloma Virus, antigens from Epstein-Barr Virus, LAGE1, Melan A, MART-1, MAGE-1, MAGE-3, BAGE, GAGE-1, GAGE-2, tyrosinase, gp100, gp75, c-erb-B2, CEA, MUC-1, CA-125, Stn, TAG-72, KSA (17-1A), PSMA, point-mutated RAS, EGF-R, VEGF, GD2, GM2, GD3, Anti-Id, CD20, CD19, CD22, CD36, Aberrant class II, B1, CD25, (IL-2R, anti-TAC), CA-125, CA19-9, PSA, GSTP1, promoter region of GSTP1, NGAL, CD97, CD 55, COX4-2, LAMA2, kallikrein 12, kallikrein 14, kallikrein 15, EPCA, G-CSF, leptin, prolactin, OPN, IGF-II, delta-catenin, ERR.gamma., hK10, hK6, hK2, alpha-haptoglobin, PKC, calreticulin, 125P5C8, Nicotinamide N-methyltransferase, ULIP proteins, ITG.beta.6, TIMP-1, Nup88, Csk autoantibodies, VEGFR, Neuropilins, COTA, hnRNP, TSC403, or NCA 50/90.

Aspect 142. The method of aspect 139, wherein said antigen is administered in the form of a cellular vaccine. Aspect

Aspect 143. The method of aspect 142, wherein said cellular vaccine is inactivated mitotically.

Aspect 144. The method of aspect 143, wherein said mitotic inactivation is accomplished by chemical fixation.

Aspect 145. The method of aspect 143, wherein said mitotic inactivation is accomplished by mitomycin C treatment.

Aspect 146. The method of aspect 143, wherein said mitotic inactivation is accomplished by irradiation.

Aspect 147. The method of aspect 142, wherein said cellular vaccine is produced by treating cells with hyperthermia for a sufficient time period to induce an increase in immunogenicity.

Aspect 148. The method of aspect 147, wherein said increase in immunogenicity is quantified by ability to stimulate a mixed lymphocyte reaction comprising of mitotically inactivated tumor cells and allogeneic lymphocytes.

Aspect 149. The method aspect 142, wherein said cellular vaccine is generated from autologous cells of the patient in need of treatment.

Aspect 150. The method of aspect 142, wherein said cellular vaccine is generated from cells expressing CD133 from a patient biopsy.

Aspect 151. The method of aspect 142, wherein said cellular vaccine is generated from a cell line.

Aspect 152. The method of aspect 151, wherein said cell line is selected from a group of cell lines comprising of: tumor cell lines are selected from a group comprising of: J82, RT4, ScaBER, T24, TCCSUP, 5637 Carcinoma, SK-N-MC Neuroblastoma, SK-N-SH Neuroblastoma, SW 1088 Astrocytoma, SW 1783 Astrocytoma, U-87 MG Glioblastoma, astrocytoma, grade III, U-118 MG Glioblastoma, U-138 MG Glioblastoma, U-373 MG Glioblastoma, astrocytoma, grade III, Y79 Retinoblastoma, BT-20 Carcinoma, breast, BT-474 Ductal carcinoma, breast, MCF7 Breast adenocarcinoma, pleural effusion, MDA-MB-134-V Breast, ductal carcinoma, pleural I effusion, MDA-MD-157 Breast medulla, carcinoma, pleural effusion, MDA-MB-175-VII Breast, ductal carcinoma, pleural Effusion, MDA-MB-361 Adenocarcinoma, breast, metastasis to brain, SK-BR-3 Adenocarcinoma, breast, malignant pleural effusion, C-33 A Carcinoma, cervix, HT-3 Carcinoma, cervix, metastasis to lymph node

ME-180 Epidermoid carcinoma, cervix, metastasis to omentum, MEL-175 Melanoma, MEL-290 Melanoma, HLA-A*0201 Melanoma cells, MS751 Epidermoid carcinoma, cervix, metastasis to lymph

Node, SiHa Squamous carcinoma, cervix, JEG-3 Choriocarcinoma, Caco-2 Adenocarcinoma, colon

HT-29 Adenocarcinoma, colon, moderately well-differentiated grade II, SK-CO-1 Adenocarcinoma, colon, ascites, HuTu 80 Adenocarcinoma, duodenum, A-253 Epidermoid carcinoma, submaxillary gland

FaDu Squamous cell carcinoma, pharynx, A-498 Carcinoma, kidney, A-704 Adenocarcinoma, kidney

Caki-1 Clear cell carcinoma, consistent with renal primary, metastasis to skin, Caki-2 Clear cell carcinoma, consistent with renal primary, SK-NEP-1 Wilms' tumor, pleural effusion, SW 839 Adenocarcinoma, kidney, SK-HEP-1 Adenocarcinoma, liver, ascites, A-427 Carcinoma, lung

Calu-1 Epidermoid carcinoma grade III, lung, metastasis to pleura, Calu-3 Adenocarcinoma, lung, pleural effusion, Calu-6 Anaplastic carcinoma, probably lung, SK-LU-1 Adenocarcinoma, lung consistent with poorly differentiated, grade III, SK-MES-1 Squamous carcinoma, lung, pleural effusion, SW 900 Squamous cell carcinoma, lung, EB1 Burkitt lymphoma, upper maxilia, EB2 Burkitt lymphoma, ovary

P3HR-1 Burkitt lymphoma, ascites, HT-144 Malignant melanoma, metastasis to subcutaneous tissue

Malme-3M Malignant melanoma, metastasis to lung, RPMI-7951 Malignant melanoma, metastasis to lymph node, SK-MEL-1 Malignant melanoma, metastasis to lymphatic system, SK-MEL-2 Malignant melanoma, metastasis to skin of thigh, SK-MEL-3 Malignant melanoma, metastasis to lymph node

SK-MEL-5 Malignant melanoma, metastasis to axillary node, SK-MEL-24 Malignant melanoma, metastasis to node, SK-MEL-28 Malignant melanoma, SK-MEL-31 Malignant melanoma, Caov-3 Adenocarcinoma, ovary, consistent with primary, Caov-4 Adenocarcinoma, ovary, metastasis to subserosa of fallopian tube, SK-OV-3 Adenocarcinoma, ovary, malignant ascites, SW 626 Adenocarcinoma, ovary, Capan-1 Adenocarcinoma, pancreas, metastasis to liver, Capan-2 Adenocarcinoma, pancreas, DU 145 Carcinoma, prostate, metastasis to brain, A-204 Rhabdomyosarcoma, Saos-2 Osteogenic sarcoma, primary, SK-ES-1 Anaplastic osteosarcoma versus Swing sarcoma, SK-LNS-1 Leiomyosarcoma, vulva, primary, SW 684 Fibrosarcoma, SW 872 Liposarcoma

SW 982 Axilla synovial sarcoma, SW 1353 Chondrosarcoma, humerus, U-2 OS Osteogenic sarcoma, bone primary, Malme-3 Skin fibroblast, KATO III Gastric carcinoma, Cate-1B Embryonal carcinoma, testis, metastasis to lymph node, Tera-1 Embryonal carcinoma, Tera-2 Embryonal carcinoma, SW579 Thyroid carcinoma, AN3 CA Endometrial adenocarcinoma, metastatic, HEC-1-A Endometrial adenocarcinoma

HEC-1-B Endometrial adenocarcinoma, SK-UT-1 Uterine, mixed mesodermal tumor, consistent with

leiomyosarcomagrade III, SK-UT-1B Uterine, mixed mesodermal tumor, Sk-Me128 Melanoma

SW 954 Squamous cell carcinoma, vulva, SW 962 Carcinoma, vulva, lymph node metastasis, NCI-H69 Small cell carcinoma, lung, NCI-H128 Small cell carcinoma, lung, BT-483 Ductal carcinoma, breast

BT-549 Ductal carcinoma, breast, DU4475 Metastatic cutaneous nodule, breast carcinoma

HBL-100 Breast, Hs 578Bst Breast, Hs 578T Ductal carcinoma, breast, MDA-MB-330 Carcinoma, breast

MDA-MB-415 Adenocarcinoma, breast, MDA-MB-435s Ductal carcinoma, breast, MDA-MB-436 Adenocarcinoma, breast, MDA-MB-453 Carcinoma, breast, MDA-MB-468 Adenocarcinoma, breast

T-47D Ductal carcinoma, breast, pleural effusion, Hs 766T Carcinoma, pancreas, metastatic to lymph node, Hs 746T Carcinoma, stomach, metastatic to left leg, Hs 695T Amelanotic melanoma, metastatic to lymph node, Hs 683 Glioma, Hs 294T Melanoma, metastatic to lymph node, Hs 602 Lymphoma, cervical

JAR Choriocarcinoma, placenta, Hs 445 Lymphoid, Hodgkin's disease, Hs 700T Adenocarcinoma, metastatic to pelvis, H4 Neuroglioma, brain, Hs 696 Adenocarcinoma primary, unknown, metastatic

to bone-sacrum, Hs 913T Fibrosarcoma, metastatic to lung, Hs 729 Rhabdomyosarcoma, left leg, FHs 738Lu Lung, normal fetus, FHs 173We Whole embryo, normal, FHs 738B1 Bladder, normal fetus

NIH:OVCAR-3 Ovary, adenocarcinoma, Hs 67 Thymus, normal, RD-ES Ewing's sarcoma

ChaGo K-1 Bronchogenic carcinoma, subcutaneous, metastasis, human, WERI-Rb-1 Retinoblastoma

NCI-H446 Small cell carcinoma, lung, NCI-H209 Small cell carcinoma, lung, NCI-H146 Small cell carcinoma, lung, NCI-H441 Papillary adenocarcinoma, lung, NCI-H82 Small cell carcinoma, lung

H9 T-cell lymphoma, NCI-H460 Large cell carcinoma, lung, NCI-H596 Adenosquamous carcinoma, lung

NCI-H676B Adenocarcinoma, lung, NCI-H345 Small cell carcinoma, lung, NCI-H820 Papillary adenocarcinoma, lung, NCI-H520 Squamous cell carcinoma, lung, NCI-H661 Large cell carcinoma, lung

NCI-H510A Small cell carcinoma, extra-pulmonary origin, metastatic D283 Med Medulloblastoma

Daoy Medulloblastoma, D341 Med Medulloblastoma, AML-193 Acute monocyte leukemia

MV4-11 Leukemia biphenotype

153. The method of aspect 139, wherein blockade of an immune inhibitory molecule is additionally performed.

154. The method of aspect 153, wherein said inhibitory molecule is selected from a group comprising of: PD1, PD-L1, CTLA4, TIM3, LAGS, VISTA, BTLA, TIGIT, LAIR1, CD160, 2B4 and TGF-beta.

155. A method of treating cancer comprising: a) extracting microvesicles from a cancer patient; b) concentrating said microvesicles; c) using said microvesicles as a source of tumor antigen for immunization; d) performing an immunopheresis procedure prior to, and/or subsequently to immunization.

Aspect 156. The method of aspect 155, wherein said microvesicles are exosomes.

Aspect 157. The method of aspect 156, wherein said exosomes possess one or more tumor antigens that are found in the tumor of the patient from which said exosomes are extracted.

Aspect 158. The method of aspect 156, wherein said microvesicles are extracted from blood.

Aspect 159. The method of aspect 156, wherein said microvesicles are extracted from lymphatic fluid.

Aspect 160. The method of aspect 156, wherein said microvesicles are extracted from ascites fluid.

Aspect 161. The method of aspect 156, wherein said microvesicles are extracted from urine.

Aspect 162. The method of aspect 156, wherein said microvesicles are extracted from semen.

Aspect 163. The method of aspect 156, wherein said microvesicles are extracted from cerebrospinal fluid.

Aspect 164. The method of aspect 156, wherein said microvesicles are allogeneic to the patient in which they are administered.

Aspect 165. The method of aspect 156, wherein said microvesicles are xenogeneic to the patient in which they are administered.

Aspect 166. The method of aspect 156, wherein said microvesicles are apoptotic bodies.

Aspect 167. The method of aspect 156, wherein said microvesicles are collected subsequent to an intervention that causes stress and/or death of tumor cells.

Aspect 168. The method of aspect 156, wherein microvesicles are concentrated by an affinity means to a molecule found on exosomes and particularly on tumor exosomes, said molecules are selected from a group comprising of: a) highly glycosylated membrane proteins; b) phosphatidylserine; and c) tetraspanin molecules

Aspect 169. The method of aspect 168, exosomes are concentrated using gradient centrifugation.

Aspect 170. The method of aspect 169, wherein said lectin is selected from the group consisting of GNA, NPA, Concanavalin A and cyanovirin.

Aspect 171. The method of aspect 167, wherein said exosomes express one or more proteins selected from a group comprising of: Fas ligand, MHC I, CD44, placental alkaline phosphatase, TSG-101, MHC I-peptide complexes, MHC II-peptide complexes, and proteins found to be present on the exterior of microvesicles contributing to immune suppression found in a cancer patient.

Aspect 172. The method of aspect 157, wherein said microvesicles are treated in a manner to increase immunogenicity.

Aspect 173. The method of aspect 172, wherein said treatment of microvesicles to increase immunogenicity is conjugation to an antibody possessing an affinity to phosphotidylserine, wherein said antibody is capable of eliciting internalization of said microvesicle by antigen presenting cells.

Aspect 174. The method of aspect 173, wherein said antibody is a bispecific antibody capable of selectively conjugating said microvesicles with said antigen presenting cell.

Aspect 175. The method of aspect 173, wherein said antibody possesses an Fc potion which allows for internalization of said microvesicle by an antigen presenting cell.

Aspect 176. The method of aspect 157, wherein said microvesicles are administered to an antigen presenting cell ex vivo and subsequently said antigen presenting cells are administered to a patient in need of therapy.

Aspect 177. A method of aspects 1-14 wherein said treatment of cancer comprises: a) administering nanoparticles for immunization and b) performing an immunopheresis procedure prior to, and/or subsequently to immunization.

Aspect 178. The method of aspect 177 wherein use of nanoparticles are for delivery of vaccine adjuvants in combination with extracorporeal reduction of immune inhibitors wherein said nanoparticles are comprised of a polymer and an immunogen.

Aspect 179. The method of aspect 178 wherein the adjuvants are selected from the following group: monophosphoryl Lipid A/synthetic trehalose dicorynomycolate (MPL-TDM), AS021/AS02, nonionic block co-polymer adjuvants, CRL 1005, aluminum phosphates, AIPO4), R-848, imiquimod, PAM3CYS, poly (I:C), loxoribine, bacille Calmette-Guerin (BCG), Corynebacterium parvum, CpG oligodeoxynucleotides (ODN), cholera toxin derived antigens, CTA 1-DD, lipopolysaccharide adjuvants, complete Freund's adjuvant, incomplete Freund's adjuvant, saponin, mineral gels, aluminum hydroxide, surface active substances, lysolecithin, pluronic polyols, polyanions, peptides, oil or hydrocarbon emulsions in water, MF59, Montanide ISA 720, keyhole limpet hemocyanins (KLH), dinitrophenol, adenosine receptor inhibitors and combinations thereof.

Aspect 180. The method of aspect 178 wherein the immunogen may be selected from a group of antigens comprising of: AMACR, PAP, PSM, MAGE, NY-ESO-1, BORIS, MUM-1, p53, CDK4, HER2/NEU, antigens from Papilloma Virus, antigens from Epstein-Barr Virus, LAGE1, Melan A, MART-1, MAGE-1, MAGE-3, BAGE, GAGE-1, GAGE-2, tyrosinase, gp100, gp75, c-erb-B2, CEA, MUC-1, CA-125, Stn, TAG-72, KSA (17-1A), PSMA, point-mutated RAS, EGF-R, VEGF, GD2, GM2, GD3, Anti-Id, CD20, CD19, CD22, CD36, Aberrant class II, B1, CD25, (IL-2R, anti-TAC), CA-125, CA19-9, PSA, GSTP1, promoter region of GSTP1, NGAL, CD97, CD 55, COX4-2, LAMA2, kallikrein 12, kallikrein 14, kallikrein 15, EPCA, G-CSF, leptin, prolactin, OPN, IGF-II, delta-catenin, ERR.gamma., hK10, hK6, hK2, alpha-haptoglobin, PKC, calreticulin, 125P5C8, Nicotinamide N-methyltransferase, ULIP proteins, ITG.beta.6, TIMP-1, Nup88, Csk autoantibodies, VEGFR, Neuropilins, COTA, hnRNP, TSC403, or NCA 50/90.

Aspect 181. The method of aspect 177-179 wherein the nanoparticles are further comprised of a targeting moiety.

Aspect 182. The method of aspect 181 wherein the targeting moiety is selected from the following group: In some aspects the nanoparticles are further comprised of targeting moieties selected from the group consisting of chitosan, mannin, mannitol, polypeptide aptamers, polynucleotide aptamers, RNA aptomers, DNA aptomers, x-aptomers, peptides, polypeptides, antibodies, antibody fragments, Fv fragments, camelids, nanobodies, ligands, RGD, fibronectin and mixtures or combinations thereof.

Aspect 183. The method of aspect 182 wherein the targeting moiety is selected to target the following group: (VCAM)-1, 30.5 kDa antigen, CD34, VEGF, VEGF-VEGFR complex, endosialin, selectins, □v integrins, endoglin, Tie 2, angiostatin receptor, MMP2/MMP9, CD13/Aminopeptidase N, endostatin receptor, TEM1/5/8, VE cadherin cryptic epitope, CD44v3, annexin A1, P-selectin, EDB-Fn, basement membrane component and mixtures or combinations thereof.

### REFERENCES

1. Page, A. R., A. E. Hansen, and R. A. Good, Occurrence of leukemia and lymphoma in patients with agammaglobulinemia. Blood, 1963. 21: p. 197-206.
2. Salavoura, K., et al., Development of cancer in patients with primary immunodeficiencies. Anticancer Res, 2008. 28(2B): p. 1263-9.
3. Kinlen, L. J., et al., Prospective study of cancer in patients with hypogammaglobulinaemia. Lancet, 1985. 1(8423): p. 263-6.
4. van der Meer, J. W., et al., Colorectal cancer in patients with X-linked agammaglobulinaemia. Lancet, 1993. 341(8858): p. 1439-40.
5. Brent, J., et al., Clinical and laboratory correlates of lung disease and cancer in adults with idiopathic hypogammaglobulinaemia. Clin Exp Immunol, 2016. 184(1): p. 73-82.
6. Bae, S. B., et al., Expression of Programmed Death Receptor Ligand 1 with High Tumor-Infiltrating Lymphocytes Is Associated with Better Prognosis in Breast Cancer. J Breast Cancer, 2016. 19(3): p. 242-251.
7. Zhang, Y., et al., Prognostic significance of immune cells in the tumor microenvironment and peripheral blood of gallbladder carcinoma patients. Clin Transl Oncol, 2016.
8. Santoiemma, P. P., et al., Systematic evaluation of multiple immune markers reveals prognostic factors in ovarian cancer. Gynecol Oncol, 2016. 143(1): p. 120-7.
9. Gonzalez, N. L., et al., Myasthenia triggered by immune checkpoint inhibitors: New case and literature review. Neuromuscul Disord, 2017. 27(3): p. 266-268.
10. Nguyen, B. H., et al., Two cases of clinical myasthenia gravis associated with pembrolizumab use in responding melanoma patients. Melanoma Res, 2016.
11. Ghaebi, M., et al., Immune regulatory network in successful pregnancy and reproductive failures. Biomed Pharmacother, 2017. 88: p. 61-73.
12. Gleicher, N., V. A. Kushnir, and D. H. Barad, Redirecting reproductive immunology research toward pregnancy as a period of temporary immune tolerance. J Assist Reprod Genet, 2017.
13. Sammar, M., et al., Expression of CD24 and Siglec-10 in first trimester placenta: implications for immune tolerance at the fetal-maternal interface. Histochem Cell Biol, 2016.
14. Engler, J. B., et al., Glucocorticoid receptor in T cells mediates protection from autoimmunity in pregnancy. Proc Natl Acad Sci USA, 2017. 114(2): p. E181-E190.
15. Ostrand-Rosenberg, S., et al., Frontline Science: Myeloid-derived suppressor cells (MDSCs) facilitate maternal-fetal tolerance in mice. J Leukoc Biol, 2016.
16. Rogovskii, V. S., The linkage between inflammation and immune tolerance: interfering with inflammation in cancer. Curr Cancer Drug Targets, 2017.
17. Lin, C. F., et al., Escape from IFN-gamma-dependent immunosurveillance in tumorigenesis. J Biomed Sci, 2017. 24(1): p. 10.
18. Chang, J. H., Y. Jiang, and V. G. Pillarisetty, Role of immune cells in pancreatic cancer from bench to clinical application: An updated review. Medicine (Baltimore), 2016. 95(49): p. e5541.
19. Wong, Y. C., et al., The CD8 T-cell response during tolerance induction in liver transplantation. Clin Transl Immunology, 2016. 5(10): p. e102.
20. Baroja-Mazo, A., et al., Tolerance in liver transplantation: Biomarkers and clinical relevance. World J Gastroenterol, 2016. 22(34): p. 7676-91.
21. Wegner, A., J. Verhagen, and D. C. Wraith, Myeloid-derived suppressor cells mediate tolerance induction in autoimmune disease. Immunology, 2017.
22. Sanjabi, S., S. A. Oh, and M. O. Li, Regulation of the Immune Response by TGF-beta: From Conception to Autoimmunity and Infection. Cold Spring Harb Perspect Biol, 2017.
23. Talotta, R., et al., Biological Agents In Rheumatoid Arthritis: A Cross-Link Between Immune Tolerance And Immune Surveillance. Curr Rheumatol Rev, 2016.
24. Alexander, T., et al., Resetting the immune system with immunoablation and autologous haematopoietic stem cell transplantation in autoimmune diseases. Clin Exp Rheumatol, 2016. 34(4 Suppl 98): p. 53-7.
25. Lan, J. L., et al., Reduced risk of all-cancer and solid cancer in Taiwanese patients with rheumatoid arthritis treated with etanercept, a TNF-alpha inhibitor. Medicine (Baltimore), 2017. 96(7): p. e6055.
26. Chang, L., et al., Irradiation enhances dendritic cell potential antitumor activity by inducing tumor cell expressing TNF-alpha. Med Oncol, 2017. 34(3): p. 44.
27. Gridley, D. S., et al., Time course of serum cytokines in patients receiving proton or combined photon/proton beam radiation for resectable but medically inoperable non-small-cell lung cancer. Int J Radiat Oncol Biol Phys, 2004. 60(3): p. 759-66.
28. Madu, M. F., et al., Isolated Limb Perfusion for Melanoma is Safe and Effective in Elderly Patients. Ann Surg Oncol, 2017.
29. Jakob, J. and P. Hohenberger, Role of isolated limb perfusion with recombinant human tumor necrosis factor alpha and melphalan in locally advanced extremity soft tissue sarcoma. Cancer, 2016. 122(17): p. 2624-32.
30. Deroose, J. P., et al., Treatment modifications in tumour necrosis factor-alpha (TNF)-based isolated limb perfusion in patients with advanced extremity soft tissue sarcomas. Eur J Cancer, 2015. 51(3): p. 367-73.
31. Ma, X., et al., Recombinant mutated human TNF in combination with chemotherapy for stage IIIB/IV non-small cell lung cancer: a randomized, phase III study. Sci Rep, 2015. 4: p. 9918.

1. A method of augmenting an existing antigen-specific immune response towards a neoplastic cell comprising the steps of: a) selecting a patient suffering from a cancer; b) identifying existing immune responses towards the cancer; c) performing one or more immunopheresis procedures subsequent to vaccinating towards antigens associated with existing antigen-specific immune response; d) optionally performing vaccination towards antigens associated with said existing antigen-specific immune response subsequent to one or more immunopheresis procedures.

2. The method of claim 1, wherein said tumor associated antigens are immunogenic peptides derived from proteins selected from a proteins comprising of: a) Fos-related antigen 1; b) LCK; c) FAP; d) VEGFR2; e) NA17; f) PDGFR-beta; g) PAP; h) MAD-CT-2; i) Tie-2; j) PSA; k) protamine 2; l) legumain; m) endosialin; n) prostate stem cell antigen; o)carbonic anhydrase IX; p) STn; q) Page4; r) proteinase 3; s) GM3 ganglioside; t) tyrosinase; u) MART1; v) gp100; w) SART3; x) RGS5; y) SSX2; z) Globol1; aa) Tn; ab) CEA; ac) hCG; ad) PRAME; ae) XAGE-1; af) AKAP-4; ag) TRP-2; ah) B7H3; ai) sperm fibrous sheath protein; aj) CYP1B1; ak) HMWMAA; al) sLe(a); am) MAGE A1; an) GD2; ao) PSMA; ap) mesothelin; aq) fucosyl GM1; ar) GD3; as) sperm protein 17; at) NY-ESO-1; au) PAX5; av) AFP; aw) polysialic acid; ax) EpCAM; ay) MAGE-A3; az) mutant p53; ba) ras; bb) mutant ras; bc) NY-BR1; bd) PAX3; be) HER2/neu; bf) OY-TES1; bg) HPV E6 E7; bh) PLAC1; bi) hTERT; bj) BORIS; bk) ML-IAP; bl) idiotype of b cell lymphoma or multiple myeloma; bm) EphA2; bn) EGFRvIII; bo) cyclin B1; bp) RhoC; bq) androgen receptor; br) surviving; bs) MYCN; bt) wildtype p53; bu) LMP2; by) ETV6-AML; bw) MUC1; bx) BCR-ABL; by) ALK; bz) WT1; ca) ERG (TMPRSS2 ETS fusion gene); cb) sarcoma translocation breakpoint; cc) STEAP; cd) OFA/iLRP; and ce) Chondroitin sulfate proteoglycan 4 (CSPG4).

3. The method of claim 1, wherein subsequent to identification of one or more antigens which are reactive to antibodies found in a patient suffering from cancer, said antigens are used for generation of a therapeutic vaccine.

4. The method of claim 3, wherein subsequent to identification of one or more antigens which are capable of stimulating T cells found in a patient suffering from cancer, said antigens are used for generation of a therapeutic vaccine.

5. The method of claim 4, wherein said antigen is administered in the form of a cellular vaccine and/or a peptide vaccine.

6. The method of claim 5, wherein said cellular vaccine is inactivated mitotically.

7. The method of claim 6, wherein said mitotic inactivation is accomplished by chemical fixation.

8. The method of claim 6, wherein said mitotic inactivation is accomplished by mitomycin C treatment.

9. The method of claim 6, wherein said mitotic inactivation is accomplished by irradiation.

10. The method of claim 5, wherein said peptide vaccine is generated from one or more peptides derived from a group of tumor antigens comprising of: a) Fos-related antigen 1; b) LCK; c) FAP; d) VEGFR2; e) NA17; f) PDGFR-beta; g) PAP; h) MAD-CT-2; i) Tie-2; j) PSA; k) protamine 2; l) legumain; m) endosialin; n) prostate stem cell antigen; o)carbonic anhydrase IX; p) STn; q) Page4; r) proteinase 3; s) GM3 ganglioside; t) tyrosinase; u) MART1; v) gp100; w) SART3; x) RGS5; y) SSX2; z) Globol1; aa) Tn; ab) CEA; ac) hCG; ad) PRAME; ae) XAGE-1; af) AKAP-4; ag) TRP-2; ah) B7H3; ai) sperm fibrous sheath protein; aj) CYP1B1; ak) HMWMAA; al) sLe(a); am) MAGE A1; an) GD2; ao) PSMA; ap) mesothelin; aq) fucosyl GM1; ar) GD3; as) sperm protein 17; at) NY-ESO-1; au) PAX5; av) AFP; aw) polysialic acid; ax) EpCAM; ay) MAGE-A3; az) mutant p53; ba) ras; bb) mutant ras; bc) NY-BR1; bd) PAX3; be) HER2/neu; bf) OY-TES1; bg) HPV E6 E7; bh) PLAC1; bi) hTERT; bj) BORIS; bk) ML-IAP; bl) idiotype of b cell lymphoma or multiple myeloma; bm) EphA2; bn) EGFRvIII; bo) cyclin B1; bp) RhoC; bq) androgen receptor; br) surviving; bs) MYCN; bt) wildtype p53; bu) LMP2; by) ETV6-AML; bw) MUC1; bx) BCR-ABL; by) ALK; bz) WT1; ca) ERG (TMPRSS2 ETS fusion gene); cb) sarcoma translocation breakpoint; cc) STEAP; cd) OFA/iLRP; and ce) Chondroitin sulfate proteoglycan 4 (CSPG4).

11. The method of claim 5 wherein an adjuvant is used together with said vaccine.

12. The method of claim 11, wherein said adjuvants are selected from the following group: monophosphoryl Lipid A/synthetic trehalose dicorynomycolate (MPL-TDM), AS021/AS02, nonionic block co-polymer adjuvants, CRL 1005, aluminum phosphates, AIPO4), R-848, imiquimod, PAM3CYS, poly (I:C), loxoribine, bacille Calmette-Guerin (BCG), Corynebacterium parvum, CpG oligodeoxynucleotides (ODN), cholera toxin derived antigens, CTA 1-DD, lipopolysaccharide adjuvants, complete Freund's adjuvant, incomplete Freund's adjuvant, saponin, mineral gels, aluminum hydroxide, surface active substances, lysolecithin, pluronic polyols, polyanions, peptides, oil or hydrocarbon emulsions in water, MF59, Montanide ISA 720, keyhole limpet hemocyanins (KLH), dinitrophenol, adenosine receptor inhibitors and combinations thereof.

13. The method of claim 1, wherein said immunopheresis is performed to remove an immune suppressive factor from patient circulation.

14. The method of claim 13, wherein said removal of an immunosuppressive device is performed by using a targeting moiety that is in contact with circulation.

15. The method of claim 14, wherein the targeting moiety is selected from the following group: In some aspects the nanoparticles are further comprised of targeting moieties selected from the group consisting of chitosan, mannin, mannitol, polypeptide aptamers, polynucleotide aptamers, RNA aptomers, DNA aptomers, x-aptomers, peptides, polypeptides, antibodies, antibody fragments, Fv fragments, camelids, nanobodies, ligands, RGD, fibronectin and mixtures or combinations thereof.

16. The method of claim 15 wherein the targeting moiety is selected to target the following group: (VCAM)-1, 30.5 kDa antigen, CD34, VEGF, VEGF-VEGFR complex, endosialin, selectins, □v integrins, endoglin, Tie 2, angiostatin receptor, MMP2/MMP9, CD13/Aminopeptidase N, endostatin receptor, TEM1/5/8, VE cadherin cryptic epitope, soluble TNF-alpha receptor I, soluble TNF-alpha receptor II, CD44v3, annexin A1, P-selectin, EDB-Fn, basement membrane component and mixtures or combinations thereof.
